(19) United States
(12) Patent Application Publication
(10) Pub. No.: US 2023/0083935 A1
(43) Pub. Date: Mar. 16, 2023

(54) METHOD AND APPARATUS FOR PARTIAL VOLUME IDENTIFICATION FROM PHOTON-COUNTING MACRO-PIXEL MEASUREMENTS

(71) Applicant: CANON MEDICAL SYSTEMS CORPORATION, Otawara-shi (JP)

(72) Inventors: Yujie LU, Vernon Hills, IL (US); Ilmar HEIN, Vernon Hills, IL (US); Zhou YU, Vernon Hills, IL (US)

(73) Assignee: CANON MEDICAL SYSTEMS CORPORATION, Otawara-shi (JP)

(21) Appl. No.: 17/469,310

(22) Filed: Sep. 8, 2021

(51) Int. Cl. G06T 11/00; G01T 1/161; A61B 6/00

(52) U.S. Cl. CPC G06T 11/005; G01T 1/161; A61B 6/4233; A61B 6/505; G06T 2210/41

(57) ABSTRACT

An apparatus and method to obtain input projection data based on radiation detected at a plurality of detector elements, reconstruct plural uncorrected images in response to applying a reconstruction algorithm to the input projection data, segment the plural uncorrected images into two or more types of material-component images by applying a deep learning segmentation network, generate output projection data corresponding to the two or more types of material-component images based on a forward projection, generate corrected multi material-decomposed projection data based on the generated output projection data corresponding to the two or more types of material-component images, and reconstruct the multi material-component images from the corrected multi material-decomposed projection data to generate one or more corrected images. In some embodiments, the plural uncorrected images are segmented into three or more types of material-component images by applying a deep learning segmentation network and beam hardening correction is performed for the three or more materials.

### FIELD OF THE INVENTION

The disclosure relates to multi-material based beam hardening correction method in computed tomography system.

### DESCRIPTION OF THE RELATED ART

Computed tomography (CT) systems and methods are widely used, particularly for medical imaging and diagnosis. CT systems generally create projection images of one or more sectional slices through a subject's body. A radiation source, such as an X-ray source, irradiates the body from one side. A collimator, generally adjacent to the X-ray source, limits the angular extent of the X-ray beam, so that radiation impinging on the body is substantially confined to a planar region (i.e., an X-ray projection plane) defining a cross-sectional slice of the body. At least one detector (and generally many more than one detector) on the opposite side of the body receives radiation transmitted through the body in the projection plane. The attenuation of the radiation that has passed through the body is measured by processing electrical signals received from the detector. In some implementations a multi slice detector configuration is used, providing a volumetric projection of the body rather than planar projections.

Typically the X-ray source is mounted on a gantry that revolves about a long axis of the body. The detectors are likewise mounted on the gantry, opposite the X-ray source. A cross-sectional image of the body is obtained by taking projective attenuation measurements at a series of gantry rotation angles, transmitting the projection data/sinogram data to a processor via the slip ring that is arranged between a gantry rotor and stator, and then processing the projection data using a CT reconstruction algorithm (e.g., inverse Radon transform, a filtered back-projection, Feldkamp-based cone-beam reconstruction, iterative reconstruction, or other method). For example, the reconstructed image can be a digital CT image that is a square matrix of elements (pixels), each of which represents a volume element (a volume pixel or voxel) of the patient's body. In some CT systems, the combination of translation of the body and the rotation of the gantry relative to the body is such that the X-ray source traverses a spiral or helical trajectory with respect to the body. The multiple views are then used to reconstruct a CT image showing the internal structure of the slice or of multiple such slices.

Most CT reconstruction algorithms assume that the x-ray source is monochromatic. In reality the x-ray source is polychromatic and the attenuation of x-rays through tissue is energy dependent. Higher energy photons are attenuated less than lower energy photons, thus the x-rays reaching the detector are “harder” than those that left the source. If not accounted for, beam hardening artifacts will appear in reconstructed images. Artifacts can include cupping as well as dark streaks and bands which can affect clinical diagnosis. A dark band present in heart muscle due to beam hardening in a cardiac scan may be interpreted as ischemia, for example. However, with the advanced CT configurations present in current scanners, beam hardening correction for clinical CT becomes more challenging. A particularly challenging case is cardiac imaging where a high-density CT contrast agent (typically iodinated contrast agent) is injected into the patient. In this case, there are multiple primary beam hardening sources and with current beam hardening methods only one or two materials of the multiple materials can be corrected, not more than two. As a result images include many artifacts that are not corrected occurring from multiple beam hardening sources.

### BRIEF DESCRIPTION OF THE DRAWINGS

A more complete understanding of this disclosure is provided by reference to the following detailed description when considered in connection with the accompanying drawings, wherein:

FIG. 1 illustrates a schematic of an exemplary computed tomography scanner;

FIG. 2A illustrates an image of a series of single energy uncorrected images used to train a segmentation network;

FIGS. 2B and 2C illustrates images of a series of labeled images from spectral CT scanning used to train a segmentation network;

FIG. 2D illustrates projections generated corresponding to different projection angles corresponding to an X-ray tube;

FIG. 3A illustrates a data flow diagram for an exemplary method of training a segmentation algorithm for multiple materials;

FIG. 3B illustrates a data flow diagram for an exemplary method of training a deep learning correction network for multiple materials;

FIG. 4 illustrates a flow chart for an exemplary method of three-dimensional multi-material based deep learning based computed tomography beam hardening correction (CT BHC);

FIG. 5A illustrates an uncorrected image before the application of three-dimensional multi-material based deep learning based CT BHC; and

FIG. 5B illustrates the uncorrected image of FIG. 5A after the application of three-dimensional multi-material based deep learning based CT BHC.

### SUMMARY

An imaging apparatus, including processing circuitry configured to obtain input projection data based on radiation detected at a plurality of detector elements, reconstruct plural uncorrected images in response to applying a reconstruction algorithm to the input projection data, segment plural the uncorrected images into two or more types of material-component images by applying a deep learning segmentation network trained to segment two or more types of material-component images, generate output projection data corresponding to the two or more types of material-component images based on a forward projection, generate corrected multi material-decomposed projection data based on the generated output projection data corresponding to the two or more types of material-component images, and reconstruct the multi material-component images from the corrected multi material-decomposed projection data to generate one or more corrected images. In some embodiments, the plural uncorrected images are segmented into three or more types of material-component images by applying a deep learning segmentation network and beam hardening correction is performed for the three or more materials.

An X-ray imaging apparatus, comprising an X-ray source configured to radiate X-rays through an object space configured to accommodate an object or subject to be imaged; a plurality of detector elements arranged across the object space and opposite to the X-ray source, the plurality of detector elements being configured to detect the X-rays from the X-ray source, and the plurality of detector elements configured to generate projection data representing counts of the X-rays, and a circuitry configured to obtain input projection data based on radiation detected at a plurality of detector elements, reconstruct plural uncorrected images in response to applying a reconstruction algorithm to the input projection data, segment the plural uncorrected images into two or more types of material-component images by applying a deep learning segmentation network trained to segment two or more types of material-component images, generate output projection data corresponding to the two or more types of material-component images based on a forward projection, generate corrected multi material-decomposed projection data based on the generated output projection data corresponding to the two or more types of material-component images, and reconstruct the multi material-component images from the corrected multi material-decomposed projection data to generate one or more corrected images. In some embodiments, the plural uncorrected images are segmented into three or more types of material-component images by applying a deep learning segmentation network and beam hardening correction is performed for the three or more materials.

An imaging method, comprising obtaining input projection data based on radiation detected at a plurality of detector elements, reconstructing plural uncorrected images in response to applying a reconstruction algorithm to the input projection data, segmenting the plural uncorrected images into two or more types of material-component images by applying a deep learning segmentation network trained to segment two or more types of material-component images, generating output projection data corresponding to the two or more types of material-component images based on a forward projection, generating corrected multi material-decomposed projection data based on the generated output projection data corresponding to the two or more types of material-component images, and reconstructing the multi material-component images from the corrected multi material-decomposed projection data to generate one or more corrected images. In some embodiments, the plural uncorrected images are segmented into three or more types of material-component images by applying a deep learning segmentation network and beam hardening correction is performed for the three or more materials.

A non-transitory computer-readable medium storing executable instructions, wherein the instructions, when executed by processing circuitry, cause the processing circuitry to perform the above-noted method.

### DETAILED DESCRIPTION

The description set forth below in connection with the appended drawings is intended as a description of various aspects of the disclosed subject matter and is not necessarily intended to represent the only aspect(s). In certain instances, the description includes specific details for the purpose of providing an understanding of the disclosed subject matter. However, it will be apparent to those skilled in the art that aspects may be practiced without these specific details. In some instances, well-known structures and components may be shown in block diagram form in order to avoid obscuring the concepts of the disclosed subject matter.

Reference throughout the specification to “one aspect” or “an aspect” means that a particular feature, structure, characteristic, operation, or function described in connection with an aspect is included in at least one aspect of the disclosed subject matter. Thus, any appearance of the phrases “in one aspect” or “in an aspect” in the specification is not necessarily referring to the same aspect. Further, the particular features, structures, characteristics, operations, or functions may be combined in any suitable manner in one or more aspects. Further, it is intended that aspects of the disclosed subject matter can and do cover modifications and variations of the described aspects.

It must be noted that, as used in the specification and the appended claims, the singular forms “a,” “an,” and “the” include plural referents unless the context clearly dictates otherwise. That is, unless clearly specified otherwise, as used herein the words “a” and “an” and the like carry the meaning of “one or more.” Additionally, it is to be understood that terms such as “top,” “bottom,” “front,” “rear,” “side,” “interior,” “exterior,” and the like that may be used herein, merely describe points of reference and do not necessarily limit aspects of the disclosed subject matter to any particular orientation or configuration. Furthermore, terms such as “first,” “second,” “third,” etc., merely identify one of a number of portions, components, points of reference, operations and/or functions as described herein, and likewise do not necessarily limit aspects of the disclosed subject matter to any particular configuration or orientation.

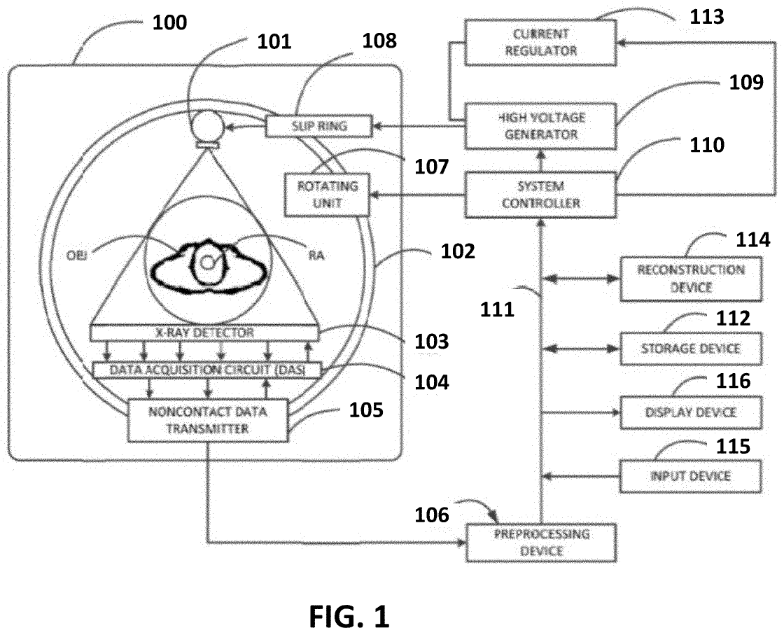

FIG. 1 shows a schematic of an implementation of a CT scanner according to an exemplary embodiment of the disclosure. Referring to FIG. 1, a radiography gantry 100 is illustrated from a side view and further includes an X-ray tube 101, an annular frame 102, and a multi-row or two-dimensional-array-type X-ray detector 103. The X-ray tube 101 and X-ray detector 103 are diametrically mounted across an object OBJ on the annular frame 102, which is rotatably supported around a rotation axis RA (or an axis of rotation). A rotating unit 107 rotates the annular frame 102 at a high speed, such as 0.4 sec/rotation, while the object OBJ is being moved along the axis RA into or out of the illustrated page.

X-ray CT apparatuses include various types of apparatuses, e.g., a rotate/rotate-type apparatus in which an X-ray tube and X-ray detector rotate together around an object to be examined, and a stationary/rotate-type apparatus in which many detection elements are arrayed in the form of a ring or plane, and only an X-ray tube rotates around an object to be examined. The techniques and components described herein can be applied to either type. The rotate/rotate type will be used as an example for purposes of clarity.

The multi-slice X-ray CT apparatus further includes a high voltage generator 109 that generates a tube voltage applied to the X-ray tube 101 through a slip ring 108 so that the X-ray tube 101 generates X-rays. The X-rays are emitted towards the object OBJ, whose cross sectional area is represented by a circle inside which a patient is illustrated. Using, the X-ray tube 101, two or more scans can be obtained corresponding to different X-ray energies. The X-ray detector 103 is located at an opposite side from the X-ray tube 101 across the object OBJ for detecting the emitted X-rays that have transmitted through the object OBJ. The X-ray detector 103 further includes individual detector elements or units.

The CT apparatus further includes other devices for processing the detected signals from X-ray detector 103. A data acquisition circuit or a Data Acquisition System (DAS) 104 converts a signal output from the X-ray detector 103 for each channel into a voltage signal, amplifies the signal, and further converts the signal into a digital signal. The X-ray detector 103 and the DAS 104 are configured to handle a predetermined total number of projections per rotation (TPPR).

The above-described data is sent to a preprocessing device 106, which is housed in a console outside the radiography gantry 100 through a non-contact data transmitter 105. The preprocessing device 106 performs certain corrections, such as sensitivity correction on the raw data. A memory 112 stores the resultant data, which is also called projection data at a stage immediately before reconstruction processing. The memory 112 is connected to a system controller 110 through a data/control bus 111, together with a reconstruction device 114, input device 115, and display 116. The system controller 110 controls a current regulator 113 that limits the current to a level sufficient for driving the CT system.

The detectors are rotated and/or fixed with respect to the patient among various generations of the CT scanner systems. In one implementation, the above-described CT system can be an example of a combined third-generation geometry and fourth-generation geometry system. In the third-generation system, the X-ray tube 101 and the X-ray detector 103 are diametrically mounted on the annular frame 102 and are rotated around the object OBJ as the annular frame 102 is rotated about the rotation axis RA. In the fourth-generation geometry system, the detectors are fixedly placed around the patient and an X-ray tube rotates around the patient. In an alternative embodiment, the radiography gantry 100 has multiple detectors arranged on the annular frame 102, which is supported by a C-arm and a stand.

The memory 112 can store the measurement value representative of the irradiance of the X-rays at the X-ray detector unit 103. Further, the memory 112 can store a dedicated program for executing, for example, various steps of the methods described herein for training and using one or more neural networks.

The reconstruction device 114 can execute various steps of methods described herein. Further, reconstruction device 114 can execute pre-reconstruction processing image processing such as volume rendering processing and image difference processing as needed.

The pre-reconstruction processing of the projection data performed by the preprocessing device 106 can include correcting for detector calibrations, detector nonlinearities, and polar effects, for example.

Post-reconstruction processing performed by the reconstruction device 114 can include filtering and smoothing the image, volume rendering processing, and image difference processing as needed. The reconstruction device 114 can use the memory to store imaging specific information, e.g., projection data, reconstructed images, calibration data and parameters, and computer programs.

The reconstruction device 114 can include a CPU (processing circuitry) that can be implemented as discrete logic gates, as an Application Specific Integrated Circuit (ASIC), a Field Programmable Gate Array (FPGA) or other Complex Programmable Logic Device (CPLD). An FPGA or CPLD implementation may be coded in VHDL, Verilog, or any other hardware description language and the code may be stored in an electronic memory directly within the FPGA or CPLD, or as a separate electronic memory. Further, the memory 112 can be non-volatile, such as ROM, EPROM, EEPROM or FLASH memory. The memory 112 can also be volatile, such as static or dynamic RAM, and a processor, such as a microcontroller or microprocessor, can be provided to manage the electronic memory as well as the interaction between the FPGA or CPLD and the memory.

Alternatively, the CPU in the reconstruction device 114 can execute a computer program including a set of computer-readable instructions that perform the functions described herein, the program being stored in any of the above-described non-transitory electronic memories and/or a hard disk drive, CD, DVD, FLASH drive or any other known storage media. Further, the computer-readable instructions may be provided as a utility application, background daemon, or component of an operating system, or combination thereof, executing in conjunction with a processor, such as a Xenon processor from Intel of America or an Opteron processor from AMD of America and an operating system, such as Microsoft VISTA, UNIX, Solaris, LINUX, Apple, MAC-OS and other operating systems known to those skilled in the art. Further, CPU can be implemented as multiple processors cooperatively working in parallel to perform the instructions.

In one implementation, the reconstructed images can be displayed on a display 116. The display 116 can be an LCD display, CRT display, plasma display, OLED, LED or any other display known in the art.

The memory 112 can be a hard disk drive, CD-ROM drive, DVD drive, FLASH drive, RAM, ROM or any other electronic storage known in the art.

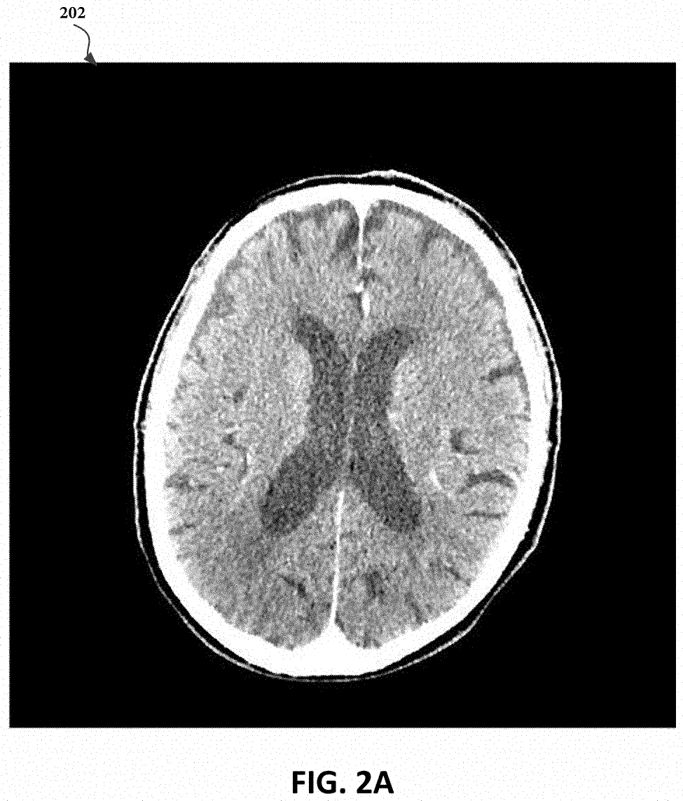

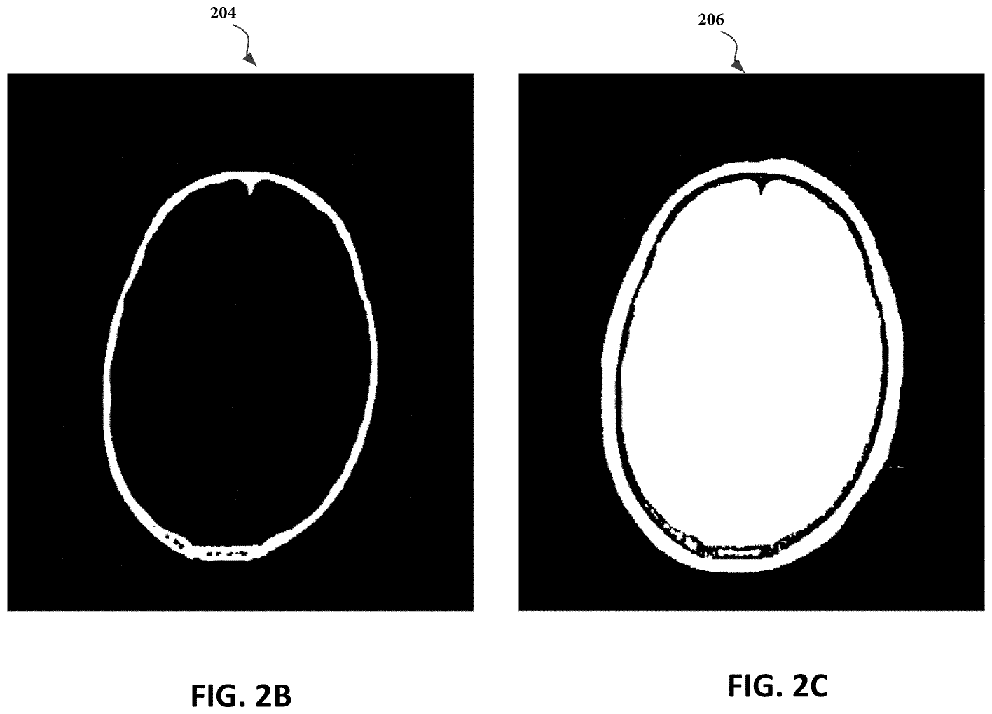

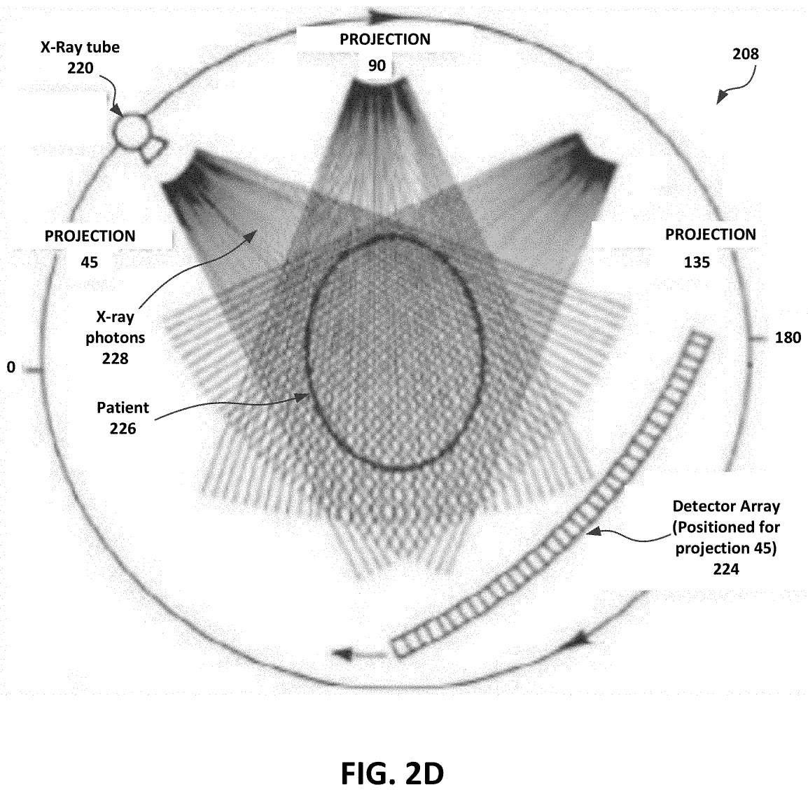

FIG. 2A shows image 202 which is an example of a single energy uncorrected image of a series of single energy uncorrected images and FIGS. 2B and 2C shows images 204 and 206 respectively that are an example of labeled images of a series of labelled images from spectral CT scanning used to train a segmentation network. Specifically, the image 202 is a conventional CT image of a brain enclosed in a skull of a patient. Further, the image 204 is an image obtained from delayed enhancements (DE) of image 202 that illustrates a bone region associated with the skull and the image 204 is labelled as a bone image. While, the image 206 is an image obtained from the DE of image 202 that illustrates a water region within the skull and the image 206 is labelled as a water image. The image 204 labelled as bone image and the image 206 labelled as water image are utilized to train the segmentation network, although any other type of labelled image may also be included to train the segmentation network.

FIG. 2D illustrates a system 208 that generates projections in accordance to different projection angles corresponding to an X-ray tube 220. The X-ray tube 220 is positioned at a projection angle of 45 degrees and a detector array 224 rotates around a patient 226. The X-ray photons 228 from the X-ray tube 220 are attenuated by the patient 226 and detected by the detector array 224 that is positioned to detect the attenuated X-ray photons 228 projected at the 45 degree projection angle of the X-ray tube 220, and generate a first projection. Similarly, the X-ray tube 220 may be positioned at a projection angle of 90 degrees or 135 degrees and accordingly corresponding projections may be obtained at the different projection angles of the X-ray tube 220.

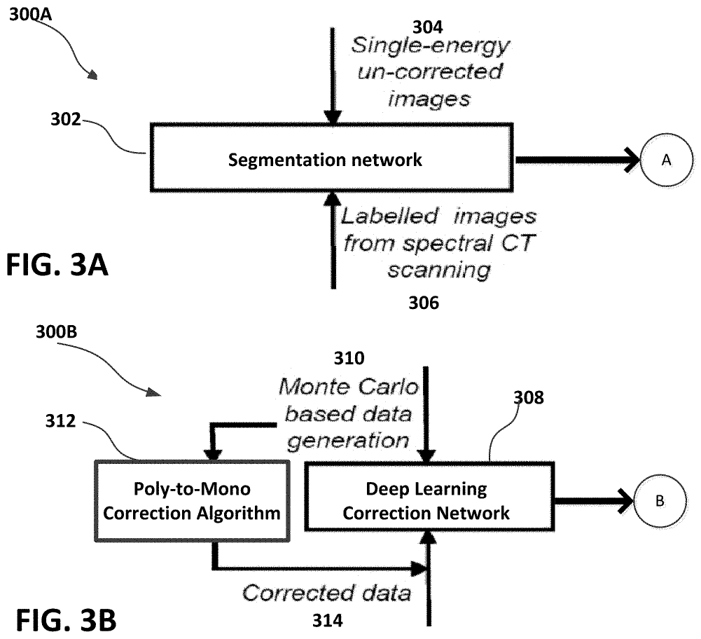

FIG. 3A shows a flow diagram 300A of a non-limiting example of a method for training a segmentation network for use in a multi-material based deep learning based computed tomography beam hardening correction (CT BHC) method.

In FIG. 3A, a portion of an offline training process is illustrated which generates a trained deep learning segmentation network for use in an on-line correction system, although other networks (e.g., 2D or 3D U-net networks or residual networks) can be used. When using a deep learning neural network as the segmentation network, the network is trained to segment images based on single energy un-corrected image data 304 (such as in FIG. 2A) as input training data and image data from spectral CT scanning 306 as the labelled data. The single energy un-corrected image data 304 includes images generated from a single polychromatic X-ray beam source with a band of energies ranging from 70 to 140 kVp (120 kVp is preferred). The single energy un-corrected image data 304 include artifacts. The labelled image data 306 (for example, as shown in FIG. 2B) is generated from dual-energy or photon counting scanning and is utilized to train the segmentation algorithm 302 to separate the uncorrected images (e.g., with overlapping Hounsfield Units (HUs) in single energy images).

The segmenting of images requires calculation of at least one attenuation coefficient. In a dual energy computed tomography, a linear attenuation coefficient is expressed by

μ(E)=(E)c₁+μ₂(E)c₂ (1)

where μ₁ (E) and μ₂ (E) are known functions of photon energy; c₁ and c₂, vary spatially, and are independent of energy. Further, μ(E) can also be expressed by

μ(E)=f(ρₑ,Z,E)+f(ρₑ,E) (2)

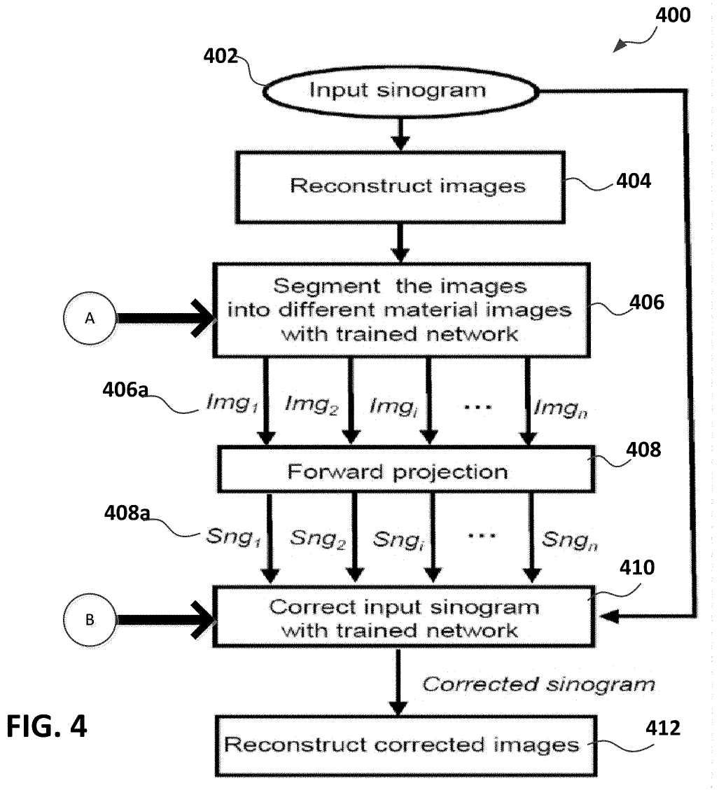

Here, ρₑ is the electron density and Z is the effective atomic number. Using the pixel based ρₑ, and Z maps, it is possible to solve the two equations using spectral information and known tissue element information. While known systems have used such techniques to segment tissue and bone, it is possible to extend that technique to larger number of materials that need to be separated (e.g., more than one contrast agent, tissue, bone, and metal internal to a body in screws, plates, etc.). Using this technique, segmented images associated with different materials including water, bone, soft tissue and iodine can be created and used as part of an offline training process to train a segmentation network. The resulting network is then used as part of an online correction process as explained in detail below in FIG. 4.

FIG. 3B shows a flow diagram 300B of a non-limiting example of a method for training a deep learning correction network for a three-dimensional multi-material based deep learning based computed tomography beam hardening correction (CT BHC) method. That is, such a network can be trained to correct input sinograms to account for beam hardening effects of many materials (rather than just two materials) that affect sinogram data. Moreover, such a network preferably also compensates for the use of multiple energy levels being used to generate the CT images instead of an idealized single energy source.

In a two-material poly-to-mono beam hardening, a corrected sinogram can be generated from an input sinogram according to:

PD_{BHC}(c,s,v)=PD_{IN}(c,s,v)+BHC3D2M(PL₁(c,s,v),PL₂(c,s,v),) (3)

where,
PL₁(c,s,v) and PL₂ (c,s,v) are the path length sinograms for the two different materials;
PD_{IN}(c,s,v) is the input sinogram;
PD_{BHC}(c,s,v) is the corrected sinogram;
c, s, and v are the index of the detector channel, segment, and projection view (as explained above with reference to FIG. 2C); and
BHC3D2M is a 4-dimensional table that acts as a correction table.

Further, the BHC3D2M can be calculated by

BHC3D2M(c,s,I₁,I₂)=log(P₀)−log(PolyCnt)−log(MonoCnt) (4)

where,

MonoCnt=I₀·e^{-l}^{₁}^{μ}^{₁}^{(mono)+l}^{₂}^{μ}^{₂}^{(mono))+••+l}^{ₙ}^{μ}^{ₙ}^{(mono))}, (5)

PolyCnt=Σ_{keV=1}^{kVP}I₀(keV)·e^{-l}^{₁}^{μ}^{₁}^{(keV)+l}^{₂}^{μ}^{₂}^{(keV)+••++l}^{ₙ}^{μ}^{ₙ}⁽keV)) (6)

P₀=Σ_{keV=1}^{kVp}I₀(keV) (7)
and l₁, l₂, ln are the path lengths of n different materials; μ₁, μ₂|•|μₙ are linear attenuation coefficient of the two materials; and I₀ is a post-wedge counts, where the post-wedge counts is a count of the X-ray source after the wedge and filters.

However, as the number of materials increases from 2 materials to n materials, the resulting correction calculation becomes:

PD_{BHC}(c,s,v)=PD_{IN}(c,s,v)+BHC3DnM(PL₁(c,s,v),PL₂(c,s,v)|••|PLₙ(c,s,v)) (8)
where,
PL₁(c,s,v), PL₂(c,s,v), PLₙ(c,s,v) are the path length sinograms for n different materials. However, since BHC3DnM is not a four dimensional correction table but rather a (n+2)-dimensional table, the size of BHC3DnM grows quickly for an increasing value of n. Therefore, it is desirable to replace the BHC3DnM correction table with a neural network that has been trained to output the corrected sinogram data based on the uncorrected sinogram data.

FIG. 3B illustrates an exemplary training configuration for a deep learning correction network 308 with the Monte Carlo based data generation method 310. The deep learning correction network 308 performs beam hardening correction for sinogram data. Training of the deep learning beam hardening correction network requires a large number of data sets for training. Accordingly, a Monte-Carlo based data generation method 310 is used to generate training data that is input to the deep learning correction network 308. In addition, to treat the generated data as mono-energy data, the training data also is input to a Poly-to-Mono correction algorithm 312, the output of which is corrected data 314 that is used as label data for the deep learning correction network 308 during training. The Monte-Carlo based data generation method 310 utilizes three random generators as follows:
1) Two independent uniform distribution generators are used to generate indices “i” and “j” for (a) a channel cᵢ and (b) a segment sⱼ, where cᵢ can take on any one of the values in the range (0, N_{chn}), and sⱼ can take on any of the values in the range (0, N_{seg}); and
2) One uniform distribution generator is used to generate the total path length (or total projection length), tlₖ where tlₖ can take on values in the range (TLₘᵢₙ, TLₘₐₓ). Further, the path lengths of different materials can be sampled using Dirichlet distribution, and is represented by

$$f ( {l}_{1} {N}_{} ; {α}_{1} {α}_{N} ) = \frac{1}{B ( α )} {\prod }_{j = 1}^{N} {l}_{j}^{{α}_{j - 1}} \qquad ( 9 )$$

where, B(α) is a normalizing constant and N is the material number, and B(α) is represented by,

$$B ( α ) = \frac{{\prod }_{j = 1}^{N} r ( {α}_{j} )}{r ( {\sum }_{j = 1}^{N} {α}_{j} )} \qquad ( 10 )$$

Upon generating, as part of the Monte-Carlo based data generation method 310, the values of a set of pathlengths of different materials from sampled channel, segment, and total pathlengths, theses values are inputted into poly-to-mono correction algorithm 312 to determine corresponding beam hardening correction values, also referred to as corrected data 314. Accordingly, upon determining the corrected values by using the poly-to-mono correction algorithm, the deep learning correction network 308 is trained using part of the data as training data and part of the data as testing data. The trained deep learning correction network 308 is then used as a component of the online correction system.

FIG. 4 shows a flow diagram 400 of a non-limiting example of a multi-material based deep learning based computed tomography beam hardening correction (CT BHC) method that utilizes the trained segmentation network of FIG. 3A and the trained deep learning correction network of FIG. 3B for beam hardening correction for two or more materials and in some embodiments three or more materials.

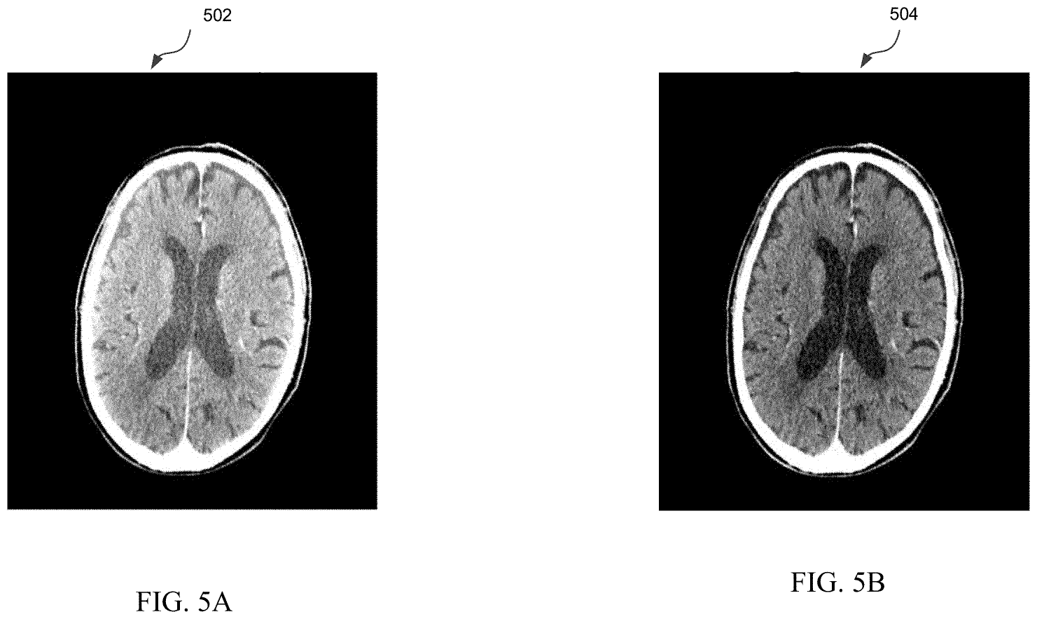

In step 402, input projection sinogram data is received from the X-ray detector 103. The input projection sinogram data includes uncorrected image sinogram data associated with a patient who is being scanned or an object that is being scanned. By way of an example, FIG. 5A illustrates an uncorrected image 502 received from the X-ray detector 103.

In step 404, the received input projection sinogram data then undergoes image reconstruction by reconstruction device 114 to generate plural reconstructed images. The reconstruction device 114 includes instructions that are executed to generate reconstructed images from the input projection sinogram data. In an embodiment, the reconstruction of the uncorrected image sinogram data is performed by applying a reconstruction algorithm (such as Feldkamp-Davis-Kress (FDK) analytic algorithm or filtered back projection (FBP) algorithm, although any other types of analytics algorithm or iterative reconstruction algorithm may also be used to the input projection sinogram data received in step 402.

In step 406, the trained segmentation network 302 of FIG. 3A is applied to the reconstructed images to segment the images. As illustrated, the trained segmentation network 302 segments the plural reconstructed images into different material images 406a represented by Img₁, Img₂, Imgᵢ, . . . , Imgₙ corresponding to the types of materials (such as soft tissue, bone, iodine, and other high-density contrast regions) that the network 302 was trained to segment.

The pathlengths of soft tissue, bone, iodine, and other high-density contrast regions are calculated by forward projection of the segmented images output in step 406. The pathlength is also referred to as projection lengths. Further, the accuracy of the pathlength calculations depends on a voxel size. The smaller the voxel size, the finer the pathlength resolution and the better the correction. Ideally, a reconstruction field of view diameter should be as small as possible and segmentation image matrix size should be as large as possible.

The voxel size is an important component of image quality, and a voxel is a 3-dimensional analog of a pixel. The voxel size is related to both the pixel size and slice thickness. The pixel size is dependent on both the field of view and the image matrix. The pixel size is equal to the field of view divided by the matrix size. The matrix size is typically 128×, 256× or 112×. Pixel size is typically between 0.5 and 1.5 mm. The smaller the pixel size, the greater the image spatial resolution.

An increased voxel size results in an increased signal-to-noise ratio. The trade-off for increased voxel size is decreased spatial resolution. The voxel size can be influenced by receiver coil characteristics. For examples, surface coils indirectly improve resolution by enabling a smaller voxel size for the same signal-to-noise ratio.

The voxel size can contribute to artifacts in MRI. Many MR artifacts are attributable to errors in the underlying spatial encoding of the radiofrequency signals arising from image voxels. The motion artifacts can occur in the phase-encoding direction because a specific tissue voxel may change location between acquisition cycles, leading to phase encoding errors. This manifests as a streak or ghost in the final image, and can be reduced with image gating and regional pre-saturation techniques.

In step 408, a forward projection algorithm is applied to the segmented images of different materials. The applied forward projection algorithm may include, but is not limited to, an X-ray tracing-based forward projection, a Footprint-based approach, and a Fast Fourier Transform (FFT)/inverse Fast Fourier Transform (i-FFT) algorithm. In this example, the X-ray tracing-based forward projection is applied to the segmented images of different materials. The X-ray is sampled at evenly spaced positions along the X-rays, and 3D interpolations of the voxels surrounding the sampling position are used as the contribution of that sampling point to the X-ray. Further, multiple forward projections are performed, for each of segmented images of different materials. The outputs of the forward projections are the pathlength sinograms 408a, PL₁[c,s,v], PL₂[c,s,v], PLi[c,s,v], . . . , PLₙ[c,s,v] corresponding to each of the segmented images of different materials. The pathlength sinograms 408a are represented by Sng₁, Sng₂, Sngᵢ, . . . , Sngₙ.
where,

Sng₁=PL₁[c,s,v],Sng₂=PL₂[c,s,v],Sng₁=PLi[c,s,v],Sngₙ=PLₙ[c,s,v]

In step 410, a trained deep learning correction network 308 of FIG. 3B is applied to correct the pathlength sinograms 408a represented in FIG. 4 by Sng₁, Sng₂, Sngᵢ, . . . , Sngₙ and output a corrected sinogram. As the trained deep learning correction network 308 is trained in FIG. 3B to correct the pathlength sinograms 408a of segmented images of different materials such as soft tissue, bone, iodine, and other high-density contrast regions, the trained deep learning correction network 308 generates corrected pathlength sinograms from the the pathlength sinograms 408a that is, in turn, reconstructed in step 412 using known methods of image reconstruction. For example, the image reconstruction process can be performed using any of a filtered back-projection method, iterative image reconstruction methods (e.g., using a total variation minimization regularization term), a Fourier-based reconstruction method, or stochastic image reconstruction methods. By way of example, FIG. 5B illustrates reconstructed corrected image 504 that can be generated in step 412. Specifically, FIG. 5B illustrates an exemplary reconstructed corrected image 504 including corrections of different materials (e.g., such as soft tissue, bone, iodine, and other high-density contrast regions) that are not seen in the uncorrected image 502.

While certain implementations have been described, these implementations have been presented by way of example only, and are not intended to limit the teachings of this disclosure. Indeed, the novel methods, apparatuses and systems described herein can be embodied in a variety of other forms; furthermore, various omissions, substitutions and changes in the form of the methods, apparatuses and systems described herein can be made without departing from the spirit of this disclosure.

According to at least one aspect of the embodiments described above, it is possible to provide an imaging apparatus, an X-ray imaging apparatus, and an imaging method.

Embodiments of the present disclosure may also be as set forth in the following parentheticals.

(1) An imaging apparatus, the imaging apparatus comprising: circuitry configured to obtain input projection data based on radiation detected at a plurality of detector elements, reconstruct plural uncorrected images in response to applying a reconstruction algorithm to the input projection data, segment the plural uncorrected images into two or more types of material-component images by applying a deep learning segmentation network trained to segment two or more types of material-component images, generate output projection data corresponding to the two or more types of material-component images based on a forward projection, generate corrected multi material-decomposed projection data based on the generated output projection data corresponding to the two or more types of material-component images, and reconstruct the multi material-component images from the corrected multi material-decomposed projection data to generate one or more corrected images.

(2) The imaging apparatus of (1), wherein the circuitry configured to generate corrected multi material-decomposed projection data comprises circuitry configured to apply a trained deep learning correction network to the output projection data corresponding to the two or more types of material-component images, wherein the trained deep learning correction network is trained to correct two or more types of material-component images.

(3) The imaging apparatus of (1) or (2), wherein the two or more types of material-component images include at least three of soft tissue, bone, water, or iodine.

(4) The imaging apparatus of (1) to (3), wherein the circuitry is further configured to determine projection lengths associated with the two or more types of material-component images, and generate the corrected multi material-decomposed projection data based at least on the determined projection lengths associated with the two or more types of material-component images.

(5) The imaging apparatus of (1) to (4), wherein the circuitry is further configured to determine a total projection length value based on the determined projection lengths associated with the two or more types of material-component images, and generate the corrected multi material-decomposed projection data based at least on the determined total projection length value associated with the two or more types of material-component images.

(6) The imaging apparatus of (2), wherein the trained deep learning correction network is trained by utilizing a poly-to-mono beam hardening correction algorithm.

(7) An X-ray imaging apparatus, the X-ray imaging apparatus comprising: an X-ray source configured to radiate X-rays through an object space configured to accommodate an object or subject to be imaged, a plurality of detector elements arranged across the object space and opposite to the X-ray source, the plurality of detector elements being configured to detect the X-rays from the X-ray source, and the plurality of detector elements configured to generate projection data representing counts of the X-rays, and a circuitry configured to obtain input projection data based on radiation detected at a plurality of detector elements, reconstruct plural uncorrected images in response to applying a reconstruction algorithm to the input projection data, segment the plural uncorrected images into two or more types of material-component images by applying a deep learning segmentation network trained to segment two or more types of material-component images, generate output projection data corresponding to the two or more types of material-component images based on a forward projection, generate corrected multi material-decomposed projection data based on the generated output projection data corresponding to the two or more types of material-component images, and reconstruct the multi material-component images from the corrected multi material-decomposed projection data to generate one or more corrected images.

(8) The X-ray imaging apparatus of (7), wherein the circuitry configured to generate corrected multi material-decomposed projection data comprises circuitry configured to apply a trained deep learning correction network to the output projection data corresponding to the two or more types of material-component images, wherein the trained deep learning correction network is trained to correct two or more types of material-component images.

(9) The X-ray imaging apparatus of (7) or (8), wherein the two or more types of material-component images include at least three of soft tissue, bone, water, or iodine.

(10) The X-ray imaging apparatus of (7) to (9), wherein the circuitry is further configured to determine projection lengths associated with the two or more types of material-component images, and generate the corrected multi material-decomposed projection data based at least on the determined projection lengths associated with the two or more types of material-component images.

(11) The X-ray imaging apparatus of (7) to (10), wherein the circuitry is further configured to determine a total projection length value based on the determined projection lengths associated with the two or more types of material-component images, and generate the corrected multi material-decomposed projection data based at least on the determined total projection length value associated with the two or more types of material-component images.

(12) The X-ray imaging apparatus of (8), wherein the trained deep learning correction network is trained by utilizing a poly-to-mono beam hardening correction algorithm.

(13) An imaging method, the method comprising: obtaining input projection data based on radiation detected at a plurality of detector elements, reconstructing plural uncorrected images in response to applying a reconstruction algorithm to the input projection data, segmenting the plural uncorrected images into two or more types of material-component images by applying a deep learning segmentation network trained to segment two or more types of material-component images, generating output projection data corresponding to the two or more types of material-component images based on a forward projection, generating corrected multi material-decomposed projection data based on the generated output projection data corresponding to the two or more types of material-component images, and reconstructing the multi material-component images from the corrected multi material-decomposed projection data to generate one or more corrected images.

(14) The method of (13), wherein the circuitry configured to generate corrected multi material-decomposed projection data comprises circuitry configured to apply a trained deep learning correction network to the output projection data corresponding to the two or more types of material-component images, wherein the trained deep learning correction network is trained to correct two or more types of material-component images.

(15) The method of (13) or (14), wherein the two or more types of material-component images include at least three of soft tissue, bone, water, or iodine.

(16) The method of (13) to (15), wherein the circuitry is further configured to determine projection lengths associated with the two or more types of material-component images, and generate the corrected multi material-decomposed projection data based at least on the determined projection lengths associated with the two or more types of material-component images.

(17) The method of (13) to (16), wherein the circuitry is further configured to determine a total projection length value based on the determined projection lengths associated with the two or more types of material-component images, and generate the corrected multi material-decomposed projection data based at least on the determined total projection length value associated with the two or more types of material-component images.

(18) The method of (14), wherein the trained deep learning correction network is trained by utilizing a poly-to-mono beam hardening correction algorithm.

(19) Any of the inventions of (1) to (18) using three or more materials instead of two or more materials.

1. An imaging apparatus, the imaging apparatus comprising:
circuitry configured to
obtain input projection data based on radiation detected at a plurality of detector elements,
reconstruct plural uncorrected images in response to applying a reconstruction algorithm to the input projection data,
segment the plural uncorrected images into three or more types of material-component images by applying a deep learning segmentation network trained to segment three or more types of material-component images,
generate output projection data corresponding to the three or more types of material-component images based on a forward projection,
generate corrected multi material-decomposed projection data based on the generated output projection data corresponding to the three or more types of material-component images, and
reconstruct the multi material-component images from the corrected multi material-decomposed projection data to generate one or more corrected images.

2. The imaging apparatus according to claim 1, wherein the circuitry configured to generate corrected multi material-decomposed projection data comprises circuitry configured to apply a trained deep learning correction network to the output projection data corresponding to the three or more types of material-component images, wherein the trained deep learning correction network is trained to correct three or more types of material-component images.

3. The imaging apparatus according to claim 1, wherein the three or more types of material-component images include at least three of soft tissue, bone, water, or iodine.

4. The imaging apparatus according to claim 1, wherein the circuitry is further configured to
determine projection lengths associated with the three or more types of material-component images, and
generate the corrected multi material-decomposed projection data based at least on the determined projection lengths associated with the three or more types of material-component images.

5. The imaging apparatus according to claim 1, wherein the circuitry is further configured to
determine a total projection length value based on the determined projection lengths associated with the three or more types of material-component images, and
generate the corrected multi material-decomposed projection data based at least on the determined total projection length value associated with the three or more types of material-component images.

6. The imaging apparatus according to claim 2, wherein the trained deep learning correction network is trained by utilizing a poly-to-mono beam hardening correction algorithm.

7. An X-ray imaging apparatus, the X-ray imaging apparatus comprising:
an X-ray source configured to radiate X-rays through an object space configured to accommodate an object or subject to be imaged,
a plurality of detector elements arranged across the object space and opposite to the X-ray source, the plurality of detector elements being configured to detect the X-rays from the X-ray source, and the plurality of detector elements configured to generate projection data representing counts of the X-rays, and
a circuitry configured to
obtain input projection data based on radiation detected at a plurality of detector elements,
reconstruct plural uncorrected images in response to applying a reconstruction algorithm to the input projection data,
segment the plural uncorrected images into three or more types of material-component images by applying a deep learning segmentation network trained to segment three or more types of material-component images,
generate output projection data corresponding to the three or more types of material-component images based on a forward projection,
generate corrected multi material-decomposed projection data based on the generated output projection data corresponding to the three or more types of material-component images, and
reconstruct the multi material-component images from the corrected multi material-decomposed projection data to generate one or more corrected images.

8. The X-ray imaging apparatus according to claim 7, wherein the circuitry configured to generate corrected multi material-decomposed projection data comprises circuitry configured to apply a trained deep learning correction network to the output projection data corresponding to the three or more types of material-component images, wherein the trained deep learning correction network is trained to correct three or more types of material-component images.

9. The X-ray imaging apparatus according to claim 7, wherein the three or more types of material-component images include at least three of soft tissue, bone, water, or iodine.

10. The X-ray imaging apparatus according to claim 7, wherein the circuitry is further configured to
determine projection lengths associated with the three or more types of material-component images, and
generate the corrected multi material-decomposed projection data based at least on the determined projection lengths associated with the three or more types of material-component images.

11. The X-ray imaging apparatus according to claim 7, wherein the circuitry is further configured to
determine a total projection length value based on the determined projection lengths associated with the three or more types of material-component images, and
generate the corrected multi material-decomposed projection data based at least on the determined total projection length value associated with the three or more types of material-component images.

12. The X-ray imaging apparatus according to claim 8, wherein the trained deep learning correction network is trained by utilizing a poly-to-mono beam hardening correction algorithm.

13. An imaging method of an improved multi-material based beam hardening correction, the method comprising:
obtaining input projection data based on radiation detected at a plurality of detector elements,
reconstructing plural uncorrected images in response to applying a reconstruction algorithm to the input projection data,
segmenting the plural uncorrected images into three or more types of material-component images by applying a deep learning segmentation network trained to segment three or more types of material-component images,
generating output projection data corresponding to the three or more types of material-component images based on a forward projection,
generating corrected multi material-decomposed projection data based on the generated output projection data corresponding to the three or more types of material-component images, and
reconstructing the multi material-component images from the corrected multi material-decomposed projection data to generate one or more corrected images.

14. The method according to claim 13, wherein the circuitry configured to generate corrected multi material-decomposed projection data comprises circuitry configured to apply a trained deep learning correction network to the output projection data corresponding to the three or more types of material-component images, wherein the trained deep learning correction network is trained to correct three or more types of material-component images.

15. The method according to claim 13, wherein the three or more types of material-component images include at least three of soft tissue, bone, water, or iodine.

16. The method according to claim 13, wherein the circuitry is further configured to
determine projection lengths associated with the three or more types of material-component images, and
generate the corrected multi material-decomposed projection data based at least on the determined projection lengths associated with the three or more types of material-component images.

17. The method according to claim 13, wherein the circuitry is further configured to
determine a total projection length value based on the determined projection lengths associated with the three or more types of material-component images, and
generate the corrected multi material-decomposed projection data based at least on the determined total projection length value associated with the three or more types of material-component images.

18. The method according to claim 14, wherein the trained deep learning correction network is trained by utilizing a poly-to-mono beam hardening correction algorithm.
